(19) United States
(12) Patent Application Publication
(10) Pub. No.: US 2025/0314667 A1
(43) Pub. Date: Oct. 9, 2025

(54) CLOSED LOOP SYSTEMS AND METHODS FOR MONITORING DIURETIC DOSAGE-REQUIREMENT

(71) Applicant: SHEBA IMPACT LTD., Ramat Gan (IL)

(72) Inventors: Ariel FURER, Ness Ziona (IL); Amitai SEGEV, Ramat Gan (IL)

(21) Appl. No.: 18/878,386

(22) PCT Filed: Jul. 5, 2023
(86) PCT No.: PCT/IL2023/050695

§ 371 Date: Dec. 23, 2024

(30) Foreign Application Priority Data
Jul. 10, 2022 (IL) 294642

(51) Int. Cl. G01N 33/84; A61B 5/20; G01N 27/30; G01N 27/414; G16H 10/60; G16H 20/17

(52) U.S. Cl. CPC G01N 33/84; A61B 5/208; G01N 27/308; G01N 27/4146; G16H 10/60; G16H 20/17; G01N 2800/325

(57) ABSTRACT

The present application provides a system for determining a dosage of a diuretic required by a subject suffering from or at risk of heart failure, which facilitates a “closed loop” of measuring urinary parameters and treating. The system comprises at least a sodium measurement sensor, such as a chemo-electrical sensor, for measuring urinary sodium levels, and one or more processors, configured to receive the measured urinary sodium levels; calculate a subject-optimized dosage of the diuretic based on the measured urinary sodium levels, optionally using a machine learning algorithm; and provide an output comprising the calculated subject-optimized dosage of diuretic. The present application further comprises methods of using the system.

### FIELD OF THE INVENTION

The present disclosure is generally directed to systems for measuring urinary sodium levels for determining diuretic dosage levels needed in cardiac patients. Specifically, the invention relates to closed loop systems for at-home or out-patient monitoring of urinary sodium levels in order to continuously determine diuretic dosage levels needed in cardiac patients.

### BACKGROUND OF THE INVENTION

Heart failure (HF) is one of the most common clinical conditions worldwide, and accordingly causes tremendous healthcare expenditure and huge operational challenges for healthcare organizations. HF affects more than 6.2 million people in the United States (US) and has a 5-year mortality rate of approximately 42%. With the prevalence expected to exceed 8 million cases by the year of 2030, projections estimate that total annual HF costs will increase to nearly US $70 billion.

One burdensome aspect of the HF problem stems from the high rate of re-hospitalizations, due to HF exacerbations, where patients are routinely admitted to the hospital to receive parenteral treatment required to overcome the acute state of congestion. 25% of HF patients are readmitted within 30 days after initial hospitalization, and 35% of these readmissions are because of HF. About half of discharged HF patients are readmitted within 6 months post discharge. This phenomenon is causing patients extreme discomfort and many times results in complications and lengthy hospital stay, way beyond the period required for the treatment of congestion.

Recently with the advent of remote monitoring systems many attempts were initiated to enable the patient to receive better care via an online support that includes a physician directing the treatment based on data gathered from different sensors as well as a conversation with the patient. Nevertheless, this sort of solution is not able to address the growing need at scale, as it still relies on the same manpower that is dire shortage and is torn between treating outpatient and hospitalized HF patients. The US alone is expected to see a shortage of up to 120,000 physicians by 2030, and cardiology is a field that will have exacerbated shortages in physician services.

One major cause for HF exacerbations and consequent rehospitalizations is the lack of adequate use of diuretics, as well as the development of diuretic resistance (i.e. the need to increase the dosage in order to get the required decongestion effect), resulting in aggravated congestion. If treated early and with an adequate dosage of drugs many of these exacerbations could be avoided or controlled thus not requiring hospitalizations. However, it is not trivial to understand the adequate dose required for treatment at every point in time according to the deterioration of congestion.

Thus, there is a need in the art for a system and method for at-home or out-patient monitoring and follow-up that enable patients to receive a dosage of diuretics that accommodates their individual and daily level of congestion. In addition, there is a need for a system and method which is independent of a physician's direct involvement with a very high safety profile.

### SUMMARY OF INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The invention relates to a closed loop system and method for continuous at-home or out-patient monitoring of urinary sodium levels and optionally urine output volume in cardiac patients, and determining, based on the sodium levels detected and optionally by using artificial intelligence (AI), appropriate dosage of diuretics needed.

In some embodiments, there is provided a system for determining a dosage of a diuretic required by a subject (subject-optimized dosage) suffering from or at risk of heart failure (HF), the system comprising a sodium measurement sensor and one or more processors, wherein:
the sodium measurement sensor is configured to measure a sodium level in a urine sample of the subject and to relay the measurement of the urinary sodium level to the one or more processors; and
the one or more processors are configured to:
   receive at least a first measurement of the urinary sodium level of the subject,
   calculate at least a first subject-optimized dosage of the diuretic based on at least the first measurement of urinary sodium level and optionally at least one medically relevant characteristic of the subject, and
   provide an output comprising at least the first calculated subject-optimized dosage of diuretic.

In some embodiments, the sodium measurement sensor is a chemo-electrical sensor. In some embodiments, the sodium measurement sensor is configured to measure only sodium levels. In some embodiments, the sodium measurement sensor comprises electronic means for transmitting signal and/or data indicating the urine sodium level to the one or more processors. In some embodiments, the sodium measurement sensor is embedded in a urine-collection cup, a condom-catheter, a diaper, or a toilet system.

In some embodiments, the system further comprises a volume sensor configured to measure urine output volume of the subject, and the one or more processors is further configured to receive at least a first urine output volume measurement, and to further calculate the subject-optimized dosage based on at least the first urine output volume measurement.

In some embodiments, the system further comprises one or more sensors configured to measure one or more urinary parameter selected from urine output volume, potassium level, chloride level, creatinine level, and osmolality, in the urine sample of the subject, and the one or more processors is further configured to receive at least a first measurement of the one or more urinary parameter, and to further calculate at least the first subject-optimized dosage based on at least the first measurement of the one or more urinary parameter.

In some embodiments, the one or more processors is further configured to receive the at least one medically relevant characteristic of the subject.

In some embodiments, the at least one medically relevant characteristic is selected from: age, gender, weight, body mass index (BMI), date of last acute exacerbation, number of previous acute exacerbations, previous dose of diuretic, type of diuretic, and any combination thereof.

In some embodiments, the one or more processors is further configured to calculate the subject-optimized dosage of the diuretic by using a machine learning algorithm.

In some embodiments, the machine learning algorithm is trained on a data set comprising dosages of diuretic administered to a plurality of subjects suffering from or at risk of heart failure, and a plurality of attributes associated with each of the plurality of dosages, the plurality of attributes comprising urinary sodium levels and optionally at least one medically relevant characteristic of the plurality of subjects.

In some embodiments, the system further comprises a user interface associated with the one or more processors, the user interface being configured to display at least the subject-optimized dosage of diuretics provided by the one or more processors, and/or to allow entering information into the processing unit.

In some embodiments, the system further comprises a delivery device functionally associated with the one or more processors, wherein the processing unit is further configured to instruct the delivery device to deliver the subject-optimized dosage of the diuretic to the subject.

In some embodiments, the delivery device is selected from a subcutaneous drug pump and a smart drug dispenser.

In some embodiments, the processing unit is further configured to calculate at least a second subject-optimized dosage of diuretics based on at least a second measurement of urinary sodium level and optionally one or more urinary parameter selected from urinary output volume, potassium level, chloride level, creatinine level, and osmolality, and on at least a first dosage of the diuretic administered to the subject after the first measurement of urinary sodium level and before the second measurement of urinary sodium level.

In some embodiments, the at least one medically relevant characteristic further comprises a change in the urinary sodium level and/or in the one or more urinary parameter between the first measurement and the second measurement.

In some embodiments, the system further comprises a urine-collecting device.

In some embodiments, there is provided a method for determining a dosage of a diuretic required by a subject (subject-optimized dosage) suffering from or at risk of heart failure, the method comprising:
taking at least a first measurement of a sodium level in a urine sample of the subject using a sodium measurement sensor;
optionally receiving at least one medically relevant characteristic of the subject;
calculating, utilizing one or more processors, at least a first subject-optimized dosage of the diuretic based on at least the first measurement of the urinary sodium level and optionally the at least one medically relevant characteristic of the subject; and
outputting at least the first calculated subject-optimized dosage of the diuretic.

In some embodiments, the method further comprises displaying the subject-optimized dosage of the diuretic on a display.

In some embodiments, the method further comprises instructing, via the one or more processors, a delivery device functionally associated with the one or more processors to deliver the subject-optimized dosage of the diuretic to the subject.

In some embodiments, the method further comprises:
taking at least a first measurement of one or more urinary parameter selected from urine output volume, potassium level, chloride level, creatinine level, and osmolality, in the urine sample of the subject using one or more additional sensors; and
further calculating the first subject-optimized dosage of the diuretic based on the at least a first measurement.

In some embodiments, the method further comprises:
taking a second measurement of sodium level and optionally one or more urinary parameter selected from urine output volume, potassium level, chloride level, creatinine level, and/or osmolality in a second urine sample of the subject obtained a predetermined time period following administration of a first dosage of the diuretic to the subject; and
further calculating a second subject-optimized dosage based on the second measurement and optionally at least one medically relevant characteristic of the subject.

In some embodiments, the at least one medically relevant characteristic of the subject includes a difference between the first measurement and the second measurement.

In some embodiments, there is provided a kit for determining a dosage of a diuretic required by a subject (subject-optimized dosage) suffering from or at risk of heart failure, the kit comprising:
a device for collecting a urine sample; and
a sodium measurement sensor configured to measure a level of sodium in the urine sample.

In some embodiments, the device is a urine-collection cup. In some embodiments, the urine-collection cup is further configured to measure a urine output volume. In some embodiments, the device is selected from a condom-like catheter, a diaper, or a pad. In some embodiments, the device comprises electronical means for transmitting signal and/or data indicating the measured urine sodium level to one or more processors.

In some embodiments, there is provided a computerized system for determining a dosage of a diuretic required by a subject (subject-optimized dosage) suffering from or at risk of heart failure (HF), comprising one or more processors configured to:
receive a measured level of urinary sodium;
calculate a subject-optimized dosage of the diuretic based at least on the measured level of urinary sodium and optionally at least one medically relevant characteristic of the subject; and
provide an output comprising at least the calculated subject-optimized dosage of diuretic.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed description.

### BRIEF DESCRIPTION OF DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1 schematically shows a block diagram of a system 100 for evaluation of diuretic dosage-requirement of a subject suffering from or at risk of heart failure, according to some embodiments.

FIG. 2 schematically shows a flow-chart of a method for optimizing a dosage of a diuretic to a subject suffering from or at risk of heart failure, according to some embodiments.

FIG. 3A schematically shows an example of a kit for optimizing a dosage of a diuretic to a subject suffering from or at risk of heart failure, provided with a urine-collection cup, according to some embodiments.

FIG. 3B schematically shows an example of a kit for optimizing a dosage of a diuretic to a subject suffering from or at risk of heart failure, provided with a diaper, according to some embodiments.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

The term “about” when referring to a measurable value such as an amount, a ratio, and the like, is meant to encompass variations of ±10% of the indicated value, as such variations are also suitable to perform the disclosed invention. Any numerical values appearing in the application are intended to be construed as if preceded by “about”, unless indicated otherwise.

Choosing the most effective dosage of diuretics by measuring urine sodium levels optionally along with urine output volume has been found to be very useful in managing heart failure (HF). Indeed, in the most updated edition of the European society of cardiology heart failure guidelines, there is a recommendation to treat hospitalized patients with acute HF and to adjust the dose based on the aforementioned parameters. However, measuring sodium level in urine samples is usually done indirectly, and requires measuring the values of sodium associated indices and deriving the sodium level from the measured indices. Accordingly, the recommendation is suitable for hospitalized patients only.

The system and method described in the present invention provide an easy way to follow parameters such as urinary sodium levels and urine output volume without the need of a health care professional or complex laboratory equipment, and to automatically derive from these parameters, and optionally administer, a dose of diuretics that is appropriate for the patient's need at the time measurements were taken. This way, patients can continuously monitor relevant urinary parameters and medicate themselves in a closed loop, without having to seek professional medical care.

Therefore, according to some embodiments, there is provided a system and a method for evaluating diuretic dosage-requirement of a subject suffering from or at risk of heart failure, based on the subject's individual and daily level of congestion. The system comprises a sodium measurement sensor, such as a chemo-electrical sensor, which is configured to measure sodium levels in a urine sample of the subject, and one or more processor configured to receive the measured level of sodium and optionally at least one medically relevant characteristic of the subject, and to calculate a subject-optimized dosage of the diuretic, optionally by applying a trained machine learning algorithm to the measured level of sodium and optionally the at least one characteristic of the subject.

The term “subject-optimized dose” or “subject-optimized dosage” is used herein to refer to a dosage of diuretics recommended for the subject, based on at least urinary sodium levels and optionally additional parameters, at the specific instance of measuring the parameters. Accordingly, the subject-optimized dosage is optimized for the subject's condition and needs at a specific time.

Advantageously, the sodium measurement sensor of the invention is configured to directly measure sodium levels in a urine sample of a subject, thereby facilitating easy determination of the sodium level in urine sample at home or in out-patient settings, without requiring the involvement of a health-care professional.

In some embodiments, there is provided a system for determining a dosage of a diuretic required by a subject (subject-optimized dosage) suffering from or at risk of heart failure (HF), the system comprising a sodium measurement sensor and one or more processors, wherein:
the sodium measurement sensor is configured to measure a sodium level in a urine sample of the subject and to relay the measurement of the urinary sodium level to the one or more processors; and
the one or more processors are configured to:
   receive at least a first measurement of the urinary sodium level of the subject,
   calculate at least a first subject-optimized dosage of the diuretic based on at least the first measurement of urinary sodium level and optionally at least one medically relevant characteristic of the subject, and
   provide an output comprising at least the first calculated subject-optimized dosage of diuretic.

In some embodiments, the at least first subject-optimized dosage of the diuretic is calculated based on at least the first measurement of urinary sodium level the and at least one medically relevant characteristic of the subject. In some embodiments, the calculating is not based on a medically relevant characteristic of the subject.

The term “dosage” as used herein with reference to a diuretic refers to the amount of the diuretic that the subject needs to take. This term is also intended to encompass a selected dosage regimen, including amount of diuretic, frequency of taking the diuretics, and mode of administration. For example, a certain dosage may refer to taking 20 mg of furosemide orally once every 12 hours.

The term “heart failure” or “HF” is used interchangeably with “congestive heart failure”, or “CHF”, and relates to a syndrome caused by an impairment of the heart's blood pumping function, which is manifested by symptoms including shortness of breath, excessive fatigue, and leg swelling.

The phrase “at risk of HF” relates to a subject who is in a risk group for having a heart failure. The subject may have suffered from HF in the past; have a family history of HF; have an underlying condition associated with HF, such as heart or blood vessel condition, lung disease, infection such as HIV, SARS, COVID, diabetes, etc.; or have a risk factor for HF, such as obesity, high blood pressure, high cholesterol, metabolic syndrome, etc.

Diuretics are regarded as the first-line treatment for patients with congestive heart failure (CHF) since they provide symptomatic relief. Diuretic medications cause increased production of urine by increasing excretion of water in the kidneys. One mechanism of action is inhibiting the reabsorption of sodium in the nephrons. The diuretic may be any diuretic suitable for treating heart failure, including loop diuretics such as furosemide (frusemide) and bumetanide, thiazide-like diuretics such as chlorothiazide, and potassium-sparing diuretics.

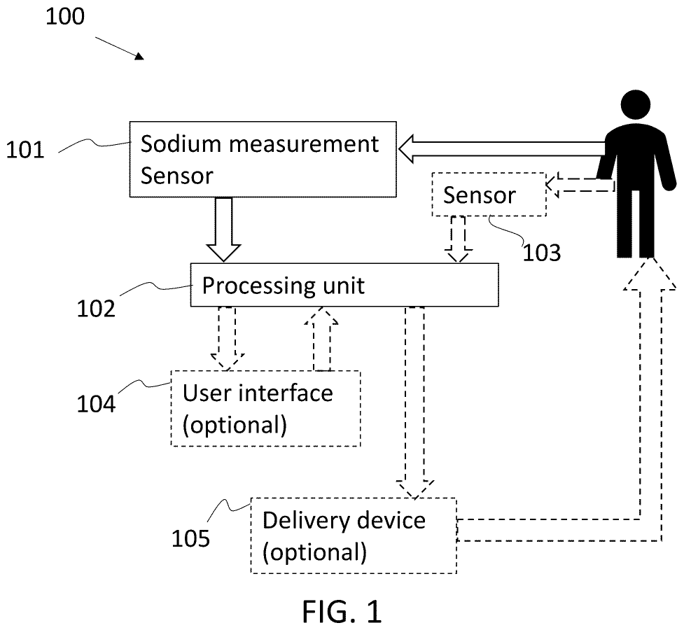

The term “processing unit” is used interchangeably herein with “one or more processor” and is intended to encompass processing circuitry on which code and calculations described herein are stored and/or executed. Since processing often takes place on remote servers or in cloud computing networks, this term is intended to encompass all places on which the code related to the system of the invention is stored and executed, regardless of whether it is physically in one place or in multiple places. Naturally, the physical processing unit is not necessarily provided with the invention, but may be provided as code (such as application) or access to such code, which exists on the remote servers or networks. However, it may be possible that a dedicated device carrying and executing the code of the invention is provided as the processing unit. Reference is now made to FIG. 1, which schematically shows a block diagram of a system 100 for determining diuretic dosage-requirement of a subject suffering from or at risk of heart failure, according to some embodiments. System 100 includes sodium measurement sensor 101, that may be a chemo-electrical sensor, which is configured to measure a level of sodium in a urine sample of the subject (termed herein “measurement of urinary sodium level”), and a processing unit (one or more processor) 102 (which may be one or more processors), configured to receive the measured level of sodium (or the measurement of urinary sodium level) and optionally at least one medically relevant characteristic of the subject, and calculate a subject-optimized dosage of the diuretic based on the measured level of sodium and optionally the at least one characteristic of the subject.

Because the system is intended for multiple sequential uses (a closed loop), the first measurement of urinary sodium level relates to a first time the processing unit receives the measurement of urinary sodium level, and the first subject-optimized dosage relates to the first subject-optimized dosage calculated by the processing unit. Similarly, a second measurement of urinary sodium level relates to a second time the processing unit receives the measurement of urinary sodium level, and the second subject-optimized dosage relates to the second subject-optimized dosage calculated by the processing unit. Additional terms are used in the same way hereinbelow. Nevertheless, for simplicity and conciseness, throughout the application such terms (as “measurement of urinary sodium level”, “subject-optimized dosage”) may be described throughout the application without using, e.g., “first” or “second” to relate to a general instance of the respective term. It is also understood that “second” is used merely as an example for further instances which are not the first instance of using the system.

In some embodiments, the processing unit 102 receives the at least one medically relevant characteristic as external input. In some embodiments, the at least one medically relevant characteristic is stored in a memory component of the processing unit (described in more detail below). In some embodiments, some of the medically relevant characteristics are received by the processing unit (e.g., inputted by a user) and some of the medically relevant characteristics are stored in the memory component of the processing unit (e.g., from previous uses of the system).

sodium measurement sensor 101 directly and accurately measures the level of sodium (Na⁺) in the urine and translates the measured level of sodium to a digital signal that is relayed to and used as the input for the processing unit 102. Accordingly, in some embodiments, the sodium measurement sensor comprises electronic means for relaying, or transmitting, signal and/or data indicating the urine sodium level to the processing unit 102. Transmission of the urine sodium level to the processing unit may be direct or indirect (e.g., by a proxy) and may be by wireless transmission or by a cable which is connectable to the sensor and to the processing unit and which is capable of transmitting information from the sensor to the processing unit.

In some embodiments, the sodium level, and levels of additional ions mentioned herein, is measured in millimole/liter or millimolar (mM). In some embodiments, the sodium level is measured in milliequivalent/liter (mEq/L). It is noted that the numerical values in mM and mEq/L are identical for sodium ions, which have a valence of 1. In some embodiments, the measurement error of the sensor does not exceed about 5%, 6%, 6.5%, or 7%. In some embodiments, the measurement error of the sensor does not exceed about 3, 4, or 5 mEq when the measurement is about 50-70 mEq/L.

In some embodiments, the sodium measurement sensor 101 is a sodium specific sensor, configured to measure only sodium levels. In some embodiments, the sodium measurement sensor is capable of measuring additional ions, such as potassium and chlorine.

In some embodiments, the sodium measurement sensor 101 is any sensor capable of measuring sodium levels in fluids such as urine. The sensor may be, e.g., a chemo-electric (or electrochemical) sensor, a spectroscopy-based sensor, or a flame photometry-based sensor.

In some embodiments, the sodium measurement sensor is a chemo-electrical sensor, such as a sensor based on ion selected electrodes (ISE). Sodium measurement sensors suitable for use with the invention include ion sensitive field-effect transistors (ISFETs), such as ISFET integrated with microfluidic interface, HfO2 gate ISFETs with and without CF4 plasma treatment, N-channel SiO2/Si3N4-gate ISFET microsensors, and 3D-extended-metal-gate ISFETs, etc., as well as printed electrodes surface-modified by carbon-based nanomaterials, metal nanomaterials, or nanocomposites (Traiwatcharanon et al., 2020, Electrochemical Sodium Ion Sensor Based on Silver Nanoparticles/Graphene Oxide Nanocomposite for Food Application, Chemosensors 8(3), 58).

In some embodiments, sodium measurement sensor 101 is a chemo-electrical sensor based on agglomeration of silver nanoparticles (AgNPs) and graphene oxide (GO) modified on a screen-printed silver electrode (SPE) for sodium detection at room temperature by using cyclic voltammetry (CV) (Traiwatcharanon et al., supra).

In some embodiments, sodium measurement sensor 101 is embedded as part of a urine-collecting device. In some embodiments, the urine-collecting device is configured to measure urine output volume. In some embodiments, the urine-collecting device is configured to measure at least a volume of 20, 50, 100, or 200 ml. In some embodiments, the device is a urine-collection cup. In some embodiments, the urine-collection cup contains at least 200 ml. In some embodiments, the urine-collection cup is graduated to allow measuring urine output volume. In some embodiments, the urine-collecting device is a condom-like catheter, which is a non-invasive tube worn by patients, collecting urine. In some embodiments, the urine-collecting device comprises wireless electronic communication means for transmitting the signal and/or data indicating the urine sodium level to the processing unit 102. In some embodiments, the urine-collecting device is disposable.me

Alternatively, in some embodiments, the sodium measurement sensor 101 is embedded as part of a smart diaper, or is assembled in a smart toilet. The last two concepts of smart diaper and smart toilet have been described in detail in the literature and are known platforms incorporating a variety of sensors, and thus may provide an appropriate setting for the implementation of sodium measurement sensor 101 described herein. In some embodiments, the smart diaper or toilet comprise wireless electronic communication means for transmitting the signal and/or data indicating the urine sodium level to the processing unit.

Sensor 103 is an optional volume sensor that measures and collects data on urine output volume, and optionally provides input in addition to that of the sodium measurement sensor 101. Therefore, according to some embodiments, system 100 further comprises a volume sensor configured to measure a urine output volume of the subject and to relay the measurement to the processing unit (in similar ways as the sodium measurement sensor), and the one or more processors is further configured to receive (at least a first) urine output volume measurement, and to further calculate the subject-optimized dosage based on the (at least first) urine output volume measurement.

It is noted that the volume sensor may be any sensor capable of providing a measurement which may be used for calculating the urine output volume even if a volume is not directly measured by the volume sensor. For example, the volume sensor may be a sensor detecting the level of fluid in a cup in which the volume of urine for each fluid level is known.

Similar to the sodium measurement sensor 101, the volume sensor 103 may also be embedded in a urine-collecting device such as a urine-collection cup or a condom-catheter, a smart diaper, or a smart toilet, as indicated above.

In some embodiments, system 100 includes both a sodium measurement sensor (such as sensor 101) and a volume sensor (such as volume sensor 103), and the one or more processors is configured to receive both the sodium levels from the sodium measurement sensor and the urine output volume from the volume sensor, and to calculate the subject-optimized level based on the urinary sodium level and the urine output volume.

In some embodiments, the volume sensor 103 is capable of accumulating readings and providing an output volume per a certain time unit, such as a daily urine output volume. Alternatively, in some embodiments, the volume sensor 103 transmits (relays) each reading (measurement) and the processor 102 calculates an output volume per time unit, such as a daily urine output volume.

In some embodiments, both the sodium measurement sensor (such as sensor 101) and a volume sensor (such as sensor 103) are embedded in a single urine-collecting device.

According to some embodiments, system 100 includes one or more additional sensors configured to measure one or more urinary parameter selected from urine output volume, potassium level, chloride level, creatinine level, and osmolality, in the urine sample of the subject, and the one or more processors is further configured to receive at least a first measurement of the one or more urinary parameter, and to further calculate at least the first subject-optimized dosage based on at least the first measurement of the one or more urinary parameter. It is noted that in some embodiments, at least one of the urinary parameters, such as, e.g., potassium ions level, is provided by the sodium measurement sensor.

According to some embodiments, the additional sensors may be embedded in a urine-collecting device such as a urine-collection cup or a condom-catheter, a smart diaper, or a smart toilet, as indicated above.

The processing unit (such as the one or more processors) 102 executes a code, which is configured to receive as input at least the measured level of sodium from the sodium measurement sensor, and optionally additional urinary parameters such as output volume and potassium level, chloride level, creatinine level, and osmolality; calculate, based at least on the input and optionally at least one characteristic of the subject, a subject-optimized dosage of the diuretic; and provide an output comprising the subject-optimized dosage. The output may be provided to a display or to another device, for allowing further actions to be taken automatically or manually, such as continued treatment, or modifying the dosage of the diuretics based on the subject-optimized dosage provided by the system.

It is appreciated that processing unit 102 includes one or more processors, and, optionally, random access memory (RAM) and/or non-volatile memory components, not shown. The one or more processors are configured to execute software instructions stored in the non-volatile memory components. Through the execution of the software instructions, measurement data (such as the measured urinary sodium level) are processed to obtain at least the calculated subject-optimal dosage of the diuretic. The measurement data may be relayed to processing unit 102 from the sodium measurement sensor 101 either directly or indirectly, e.g. via one or more intermediate agents (such as an outside device).

In some embodiments, the processing unit is further associated with at least one memory component. In some embodiments, the memory component is included in the processing unit. In some embodiments, the memory component associated with the system is not stored on the same device as the processing unit. Information may be stored in the memory component in any suitable way and on any suitable device, such as on the same device on which the processing unit is stored, or on an external device, such as cloud-based storage, a network, or a smartphone. For the purposes of the present invention, the memory component is considered to be a part of the processing unit.

The memory component enables the system to use data stored in the memory component, such as data entered by a user (e.g., the subject), or data stored in the memory component from previous instances of using the system, including, e.g., previous measurements of urine parameters and corresponding calculated subject-optimized dosages administered to the subject (or actual dosages administered to the subject).

According to some embodiments, the at least one medically relevant characteristic is selected from: age, gender, weight, body mass index (BMI), date of last acute exacerbation, number of previous acute exacerbations, previous dose of diuretic, type of diuretic, and any combination thereof.

The processing unit may receive the medically relevant characteristic by any suitable means. In some embodiments, the medically relevant characteristic(s) are inputted into the system by a user, such as the subject, or another person helping the subject. In some embodiments, the medically relevant characteristic(s) are received from an external device. In some embodiments, the medically relevant characteristic is stored in the memory component (such as from previous instances of using the system) and therefore available to the processing unit. When stating that the processing unit “receives” medically relevant parameters or other data, it is intended to include receiving the medically relevant parameters or other data from the memory component.

The processing unit calculates, based at least on the measured level of sodium and optionally the at least one characteristic of the subject, at least a subject-optimized dosage of diuretics that is recommended for the subject at the time of measurement.

According to some embodiments, the calculating includes heuristics and references.

The references may include, for example, dosages that are the standard-of-care, to start from if no other dosage is provided. For example, if the at least one medically relevant characteristic is not provided or does not include previous dose of a diuretic (for example, when starting treatment with the diuretic), then a standard-of-care dosage may be provided as a starting point, for example, 20-40 mg furosemide every 24 hours.

The heuristics may be based on published guidelines on determining dosage of diuretics. For example, the heuristics may define a level of a diuretic that should be administered based on a certain combination of urinary sodium level and urinary output. One example is adapted from the 2021 ESC Guidelines for the diagnosis and treatment of acute and chronic heart failure, European Heart Journal (2021) 42, 35993726, providing that when following administration of 20-40 mg furosemide, the urinary sodium level after 2 hours are ≥50 mEq/L, and the urine output after 6h is ≥100 mL/hour, the same dose should be repeated every 12 hours, and if the levels are lower than the reference, then the furosemide dose is doubled until the maximum is reached.

In some embodiments, the heuristics are further developed based on previous measurements or additional data.

According to some embodiments, the processing unit is configured to use a machine learning algorithm.

According to some embodiments, the machine learning algorithm is trained on a data set comprising dosages of diuretic administered to a plurality of subjects suffering from or at risk of heart failure, and a plurality of attributes associated with each of the plurality of dosages.

The plurality of attributes comprises urinary sodium level measured, optional additional measurements such as urine output volume and other urinary parameters as defined above, and optionally at least one medically relevant characteristic of the plurality of subjects. The medically relevant characteristic may for example be: age, gender, weight, BMI, date of last acute exacerbation, number of previous acute exacerbations, previous dose of diuretic, type of diuretic, or any combination thereof.

According to some embodiments, the machine learning algorithm utilized may include, for example, k-nearest neighbors (KNN), support vector machine (SVM), random forest, artificial neural networks (ANN), genetic programming (GP), linear and non-liner discriminative analysis (LDA), adaptive boosting (ADA-Boost), logistic regression, fuzzy logic, linear of non-linear kernels, reinforcement learning, or any combination thereof.

According to some embodiments, the processing unit code may be executed as a dedicated application installed on a computerized device such as a smartphone, computer, tablet, laptop or any other computerized device.

According to some embodiments, the machine learning algorithm may be executed locally on the computerized device or on a cloud computing network, or on a remote server.

In some embodiments, the machine learning algorithm is not stored as part of the processing unit code, and instead, the calculating code calls the machine learning algorithm which is stored elsewhere, such as on a cloud computing network, or on a remote server.

According to some embodiments, processing unit 102 provides a digital signal with an output comprising at least a subject-optimized dosage, and optionally a treatment regimen and/or optionally additional drugs or treatments recommended for the subject.

The output from the processing unit may be provided to various devices or components of the system, or to external devices. For example, the output may be provided to a user interface and/or to a delivery device, as described below in more detail. In some embodiments, the output is stored in the memory component. The output may be stored in the memory component in addition to being provided to additional devices or components.

In some embodiments, as discussed below with reference to the method of the invention, after a first subject-optimized dosage is provided based on the first measurement by the system of the invention, the diuretic is administered to the subject (manually or automatically), and a second measurement is taken by the system, which may include the same urinary parameters or different urinary parameters. This process of taking a measurement, calculating and providing a subject-optimized dosage, and administering the diuretics to the subject, may be repeated multiple times, with a third, fourth, etc. iterations including measurements and diuretic administration, e.g., until the HF is resolved, i.e., decongestion is achieved, and/or treatment is no longer needed.

This is the essence of the closed loop system, which facilitates urine parameters measurements and subsequent administration of dosage of diuretic needed at that time, and then follow-up measurements, and adjustments of dosage as needed, potentially until the HF is resolved.

The second measurement and any following measurements may be conducted at one or more predetermined times following administration of the diuretic. The predetermined times may be any suitable predetermined times. In some embodiments, the predetermined times are about 1-10 hours, or about 2-6 hours following administration. In some embodiments, the predetermined times are at least about 2 hours following administration. In some embodiments, the predetermined times are up to about 6 hours following administration. In some embodiments, the predetermined times are about 1, 2, 3, 4, 5, 6, 7, 8, 9, or 10, hours following administration.

In some embodiments, the dosage of diuretic administered is different from the subject-optimized dosage calculated by the system.

According to some embodiments, the processing unit is further configured to calculate at least a second subject-optimized dosage based on at least the second measurement of a urinary sodium level and optionally one or more urinary parameter selected from urinary output volume, potassium level, chloride level, creatinine level, and osmolality, and on at least the first subject-optimized dosage calculated and administered to the subject after the first measurement and before the second measurement. In case the dosage administered to the subject was different from the calculated subject-optimized dosage, the actual dosage administered may be included in the calculation instead of, or in addition to, the calculated subject-optimized dosage.

In some embodiments, the second subject-optimized dosage is calculated, similar to the first subject-optimized dosage, based on the second measurement and medically relevant parameters, and not based on the first subject-optimized dosage. In some embodiments, the medically relevant parameters comprise the first subject-optimized dosage. Optionally, the processing unit is configured to optimize the subject-optimized dosage calculation based on the change in the response of the subject to the second, as compared to first, calculated optimized dosage. Advantageously, measuring a further sodium level and optionally further medically relevant characteristics in further urine samples of the subject (such as changes in urine output volume and electrolytes following the previous diuretic dose) creates a closed loop system, which provides continuous care served with inputs intermittently.

According to some embodiments, the at least one medically relevant characteristic comprises a value calculated based on previous measurements. For example, medically relevant characteristic may be a difference (or a change) between two consecutive measurements, which provides information on the effect of the dosage of diuretics administered between the two consecutive measurements. In some embodiments, the at least one medically relevant characteristic comprises a change in the urinary sodium level and/or in the one or more urinary parameter between the first measurement and the second measurement. According to some embodiments, the at least one medically relevant characteristic further comprises a change in the urinary sodium level and/or in the one or more urinary parameter between any two consecutive measurements.

Accordingly, in some embodiments, the calculation of the subject-optimized dosage of diuretics, when conducted after at least one administration of the diuretics, takes into account the effect of at least one of the previous administrations of the diuretics on the urinary parameters, and adjusts the next subject-optimized dosage accordingly.

In some embodiments, the processing unit is further configured to take into account the effect of at least one previous administration of the diuretics on the urinary parameters, by calculating the difference between two consecutive measurements, one before administration and one after administration, and adjust the calculated subject-optimized dosage accordingly.

According to some embodiments, the sodium measurement sensor 101 is configured to measure the second level of sodium in the second urine sample of the subject. The second urine sample is obtained a predetermined time period after administration of the subject-optimized dosage of the diuretic. The processing unit receives the second level of sodium measured in the second urine sample and optimizes the calculation based on the measured second level of sodium. In case of a second level of sodium measured in a second urine sample, a further medically relevant characteristic inputted to the processing unit may be a change in urine output volume and/or electrolytes following the previous diuretic dosage administered to the subject.

According to some embodiments, sodium measurement sensor 101 is configured to measure a plurality of sodium levels in a plurality of urine samples of the subject (e.g. 2, 3, 4, 5 or more urine samples). Each sample of urine is obtained a predetermined time period after administration of the subject-optimized previous dosage of the diuretic, and the processing unit receives the level of sodium measured in each of the plurality of urine samples and optimizes the calculation based on the measured level of sodium in each of the plurality of urine samples.

According to some embodiments, system 100 further comprises a measuring device (not shown), such as a urine-collection cup for measuring a urine output volume. In some embodiments, the urine-collection cup contains at least 200 ml. Additionally or alternatively, system 100 may include a volume sensor 103 described above, which is configured to measure a urine output volume of the subject and transmit the measured output volume of urine to processing unit 102, which in turn calculates the recommended dosage of the diuretic. The urine output volume may be measured at least once, e.g., in the first urine sample, or it may be measured in all the urine samples or only in part of the urine samples. Measuring urine output volume may be done by any appropriate device and not necessarily by the sensor of the system of the invention. In some embodiments, the urine output volume is measured manually, e.g., by the subject, and manually input into the processing unit.

In some embodiments, the processing unit is associated with a user interface, such as user interface 104, optionally forming part of system 100. User interface 104 is configured to receive an output from processing unit 102, and display the output to the subject, e.g., to provide the subject with guidance of how many pills of diuretic and optionally other drugs to take.

Additionally or alternatively, according to some embodiments, user interface 104 is configured to allow a user (e.g., the subject) to input data, and relay the data received from the user, directly or indirectly to processing unit 102, by electronic means such as wireless communication or through a cable. Such data may comprise, e.g., the at least one medically relevant characteristic, and optionally data related to previous urinary measurements conducted prior to using the system of the invention. According to some embodiments, the user interface 104 is configured to display output data such as the subject-optimized dosage. In some embodiments, the user interface is configured to both display output data to a user and receive input data from a user.

The user interface 104 may be a touch screen, or alternatively it may be another type of user interface, such as a keyboard and mouse connected to a display, a smartphone, or a dedicated user interface. In addition, the user interface may be an audio user interface receiving a sound sample of the subject (for example a recording of the subject) or the like and it may provide a sound indicator for the subject regarding the subject-optimized diuretic dosage recommended to the subject.

According to some embodiments, system 100 also comprises a delivery device 105 functionally associated with processing unit 102. In some embodiments, the processing unit is configured to provide an output comprising the subject-optimized dosage, and to instruct the delivery device 105 to deliver the subject-optimized dosage of the diuretic to the subject. In some embodiments, the delivery device 105 may deliver the subject-optimized dosage of the diuretic to the subject.

The delivery device may be any suitable device capable of receiving input from a processor and administering medication to a subject. According to some embodiments, the delivery device is selected from a subcutaneous drug pump and a smart drug dispenser.

In some embodiments, instead of a delivery device which provides for automated delivery, the outputted subject-optimized dosage is manually administered to the subject.

It is noted that although the system is intended for at-home or out-patient monitoring by a subject or a care provider, it is also suitable for use in a hospital environment.

In some embodiments, there is provided a method for determining a dosage of a diuretic required by a subject (subject-optimized dosage) suffering from or at risk of heart failure. The method comprises:
measuring at least a first level of sodium in a urine sample of the subject using a sodium measurement sensor;
optionally receiving at least one medically relevant characteristic of the subject;
calculating, utilizing a processing unit, at least a first subject-optimized dosage of the diuretic based on at least the first measured level of sodium and optionally the at least one medically relevant characteristic of the subject; and
outputting at least the first calculated subject-optimized dosage of the diuretic.

In some embodiments, the method comprises receiving at least one medically relevant characteristic of the subject and calculating the at least a first subject-optimized dosage of the diuretic based on at least the first measured level of sodium and the at least one medically relevant characteristic of the subject. In some embodiments, the method does not comprise receiving at least one medically relevant characteristic of the subject and the calculating of the at least a first subject-optimized dosage of the diuretic is not based on a medically relevant characteristic of the subject.

It is appreciated that the methods of the invention described below are methods for using the systems of the invention (described above). Accordingly, the embodiments described with reference to the system also apply to the methods, mutatis mutandis.

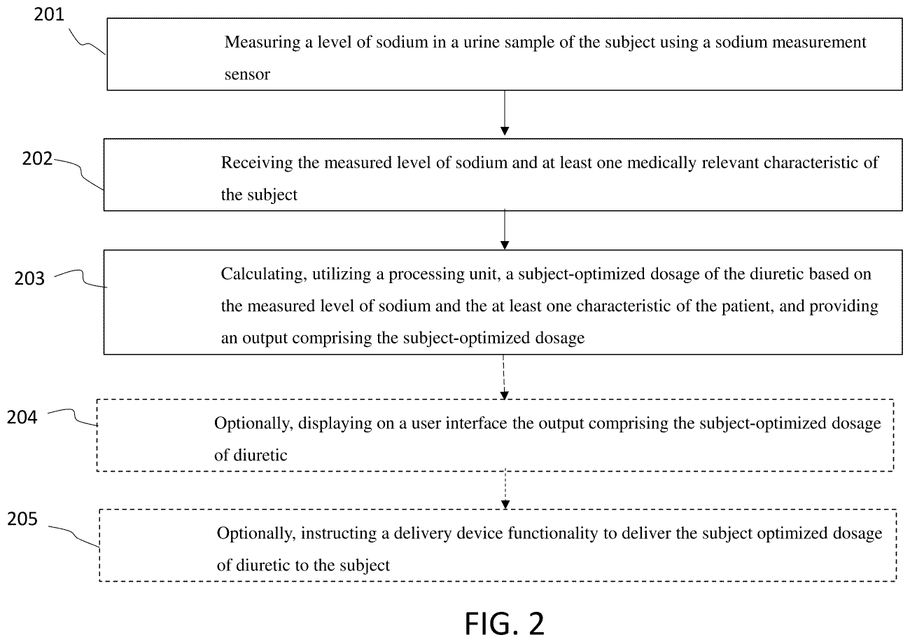

Reference is now made to FIG. 2, which schematically shows a flow-chart of a method for optimizing a dosage of a diuretic to a subject suffering from or at risk of heart failure, according to some embodiments. In step 201, a sodium measurement sensor 101, such as chemo-electrical sensor, measures a level of sodium in a urine sample of the subject. In step 202, a processing unit, such as processing unit 102, receives the measured level of sodium and optionally at least one medically relevant characteristic of the subject as an input. In step 203, the processing unit calculates the subject-optimized dosage of the diuretic based on the measured level of sodium and optionally the at least one characteristic of the subject. The calculation optionally uses a machine learning algorithm that is trained on a data set comprising dosages of diuretic administered to a plurality of subjects suffering from or at risk of heart failure, and a plurality of attributes associated with each of the plurality of dosages, and optionally uses heuristics, as explained above.

In some embodiments, the processing unit is one or more processors.

The plurality of attributes comprises urinary sodium levels measured, optional additional measurements such as urine output volume and other urinary parameters as defined above, and optionally at least one medically relevant characteristic of the plurality of subjects. The medically relevant characteristic may be for example: age, gender, weight, body mass index (BMI), date of last acute exacerbation, number of previous acute exacerbations, previous dose of diuretic, type of diuretic or any combination thereof.

According to some embodiments, the medically relevant characteristics are inputted manually, e.g., by a user, such as the subject. In some embodiments, the medically relevant characteristics are inputted through another device which is capable of connecting to and sending input to the processing unit. In some embodiments, the medically relevant characteristics are obtained from previous measurements or instances of using the system (such as previous levels of sodium or previous subject-optimized dosages) stored in the memory component. In some embodiments, the medically relevant characteristics are values calculated by the processing unit based on such previous measurements, e.g., the difference between two consecutive measurements, which provides information on the effect of the dosage of diuretics administered between the two consecutive measurements.

Obtaining data from previous instances of using the system is enabled by the optional memory component described above, which is configured to store input and/or output data related to each instance of using the system, in addition to other information, e.g., medically relevant characteristics of the subject.

According to some embodiments, calculating the subject-optimized dosage involves using a machine learning algorithm.

The machine learning algorithm may be executed locally on the one or more processors, or on a cloud computing network by a remote server. In some embodiments, the machine learning algorithm is stored on the one or more processors. In some embodiments, the machine learning algorithm is stored in a remote location and is called from the one or more processors during calculation. According to some embodiments, the machine learning algorithm utilized may be for example, k-nearest neighbors (KNN), support vector machine (SVM), random forest, artificial neural networks (ANN), genetic programming (GP), linear and non-liner discriminative analysis (LDA), adaptive boosting (ADA-Boost), logistic regression, linear of non-linear Kernals, fuzzy logic, neural network, reinforcement learning, or any combination thereof.

Following calculation of the subject-optimized dosage, the processing unit provides an output comprising the calculated subject-optimized dosage of the diuretic.

Optionally, in step 204, the output comprising the subject-optimized dosage of diuretic is displayed on a user interface unit, such as user interface 104, to provide guidance to the subject (e.g. advising the user regarding how many pills to take).

According to some embodiments, optionally, in step 205, the processing unit sends output to a delivery device functionality associated with a processing unit, such as delivery device 105 associated with processing unit 102, instructing the delivery device to deliver the subject-optimized dosage of the diuretics to the subject. The output may be transmitted directly or indirectly, by wire(s) (cables) to the processing unit, or wirelessly, such as by WiFi, Bluetooth, ZigBee and the like.

According to some embodiments, the method further comprises measuring at least a first measurement of urine output volume of the subject and inputting the measured urine output volume to the processing unit. According to some embodiments, a urine output volume of the subject is measured, e.g., by a urine-collection cup or by a measuring sensor. In some embodiments, the measured urine output volume is inputted manually by a user, such as the subject, through the user interface. In some embodiments, the measured urine output volume is inputted automatically by transmitting the measured value from the one or more sensor to the processing unit.

According to some embodiments, the method further comprises measuring at least a first measurement of one or more urinary parameters such as urine output volume, potassium level, chloride level, creatinine level, and/or osmolality in the urine sample of the subject by using one or more additional sensors, and calculating at least the first subject-optimized dosage of the diuretic by further including at least the first measurement of the one or more urinary parameters.

The measurements may be inputted manually by the subject via the user interface or alternatively the measurements may be automatically inputted by transmitting the measured values from the sensors to the processing unit directly or indirectly via electronic means, in a wired or wireless manner.

In some embodiments, the length of time from measuring the level of sodium in the urine sample until outputting the calculated subject-optimized dosage of the diuretic is no more than about 5, 10, or 15 minutes.

As long as the heart congestion is not resolved after the first administration of diuretics, there is a need to follow up the treatment in order to evaluate whether the treatment with the diuretic is effective, or whether it needs to be adjusted.

Accordingly, in some embodiments, after a first dosage of a diuretic has been administered to the subject, the method further comprises taking a second measurement of sodium level and optionally one or more urinary parameter selected from urine output volume, potassium level, chloride level, creatinine level, and/or osmolality in a second urine sample of the subject obtained a predetermined time period following the administration. A second subject-optimized dosage of the diuretics is calculated based on the second measurement and optionally on medically relevant characteristics, which may include the first dosage of the diuretics administered.

In some embodiments, the dosage of diuretic administered is different from the subject-optimized dosage calculated by the system.

In some embodiments, the medically relevant parameters include the first subject-optimized dosage, or the dosage of diuretic actually administered to the subject (if different from the subject-optimized dosage).

When a second measurement has been taken, the calculation may be optimized based on the second measurement. In addition, the medically relevant characteristics may further include a difference in sodium level and optionally one or more urinary parameter between the measurement before (e.g. the first measurement) and the measurement after (e.g., the second measurement) administration of the subject-optimized dosage of the diuretic to the subject. Alternatively, the calculation of the subject-optimized dosage after at least one administration of the diuretic may include calculating values based on previous measurements (such as levels of sodium and/or optionally other urinary parameters) and previous calculated dosages (such as the subject-optimized dosage of diuretic calculated based on certain levels of sodium and/or other urinary parameters) to improve the next subject-optimized dose.

This process of taking a measurement, calculating a subject-optimized dosage based on the latest measurement and optionally at least medically-relevant characteristics, and administering the diuretics to the subject according to the newly calculated subject-optimized dosage, may be repeated multiple times, until the HF is resolved, i.e., decongestion is achieved and treatment is no longer needed. The subject-optimized dosage is updated at each iteration based on the latest measurements, and optionally medially relevant characteristics.

Therefore, according to some embodiments, a plurality of sodium levels and optionally one or more urinary parameter are be measured in a plurality of urine samples of the subject. Each sample of urine (expect possibly for the first sample) is obtained a predetermined time after administration of the latest subject-optimized dosage of the diuretic.

The predetermined times may be any suitable predetermined times for measuring the urinary parameters in order to see an effect of the treatment. In some embodiments, the predetermined times are about 1-10 hours, or about 2-6 hours following administration. In some embodiments, the predetermined times are at least about 2 hours following administration. In some embodiments, the predetermined times are up to about 6 hours following administration. In some embodiments, the predetermined times are about 1, 2, 3, 4, 5, 6, 7, 8, 9, or 10, hours following administration.

As for the case of two measurements, the processing unit receives sodium levels and optionally one or more urinary parameter measured in each one of the plurality of urine samples and optimizes the calculation, considering the changes in values of parameters measured.

According to some embodiments, urine output volume is measured at least once, e.g., in the first urine sample. According to some embodiments, urine output volume is measured in each one of the urine samples. According to some embodiments, urine output volume is measured only in a portion of the urine samples.

In some embodiments, there is provided a method for treating a subject suffering from or at risk of heart failure, comprising:
determining a dosage of a diuretic required by the subject (subject-optimized dosage) according to the following steps:
   measuring at least a first level of sodium in a urine sample of the subject using a sodium measurement sensor;
   optionally receiving at least one medically relevant characteristic of the subject;
   calculating, utilizing a processing unit, at least a first subject-optimized dosage of the diuretic based on at least the first measured level of sodium and optionally the at least one medically relevant characteristic of the subject; and
   outputting at least the first calculated subject-optimized dosage of the diuretic, and treating the subject by administration of the calculated subject-optimized dosage.

In some embodiments, the method comprises receiving at least one medically relevant characteristic of the subject and calculating the at least first subject-optimized dosage of the diuretic based on at least the first measured level of sodium and the at least one medically relevant characteristic of the subject. In some embodiments, the method does not comprise receiving at least one medically relevant characteristic of the subject and the calculating of the at least first subject-optimized dosage of the diuretic is not based on a medically relevant characteristic of the subject.

In some embodiments, the administration is conducted by instructing, via the one or more processors, a delivery device functionally associated with the one or more processors to deliver the calculated subject-optimized dosage to the subject. In some embodiments, the administration is conducted by manually administering the calculated subject-optimized dosage to the subject.

In some embodiments, there is provided a kit for determining a dosage of a diuretic to a subject suffering from or at risk of heart failure. The kit comprises: a device for collecting a urine sample, and a sodium measurement sensor configured to measure a level of sodium in the urine sample.

The kit is intended to encompass the physical parts of the system, and therefore, in some embodiments, the kit is configured to be used together with a processing unit comprising code configured to determine the dosage of the diuretic based on at least sodium levels measured in the urine sample and optionally a medically relevant characteristic, such as an application on a smartphone, or other software on a server or network, as described above with reference to the processing unit of the system of the invention.

Accordingly, it is clarified that the sodium measurement sensor is the same sodium measurement sensor described above with reference to the system of the invention. Additionally, embodiments describing additional optional physical parts of the system, such as, e.g., sensors, also apply here. Nevertheless, some specific embodiments are described below.

In some embodiments, the kit further comprises a software application, or access to a software application, which is configured to determine the dosage of the diuretic based on at least sodium levels measured in the urine sample and optionally a medically relevant characteristic, such as an application on a smartphone, or other software on a server or network, as described above with reference to the processing unit of the system of the invention.

In some embodiments, the sodium measurement sensor is a chemo-electrical sensor. In some embodiments, the sodium measurement sensor is a chemo-electrical sensor based on agglomeration of silver nanoparticles (AgNPs) and graphene oxide (GO) modified on a screen-printed silver electrode (SPE) for sodium detection at room temperature by using cyclic voltammetry (CV).

According to some embodiments, the sodium measurement sensor includes electronic means for transmitting signal and/or data indicating the urine sodium level to a processing unit. Additionally, or alternatively, the device for collecting the urine sample in which the sodium measurement sensor may be embedded, optionally includes wireless electronic communication means for transmitting the signal and/or data indicating the urine sodium level to a processing unit.

According to some embodiments, the kit is for use together with a processing unit, such as processing units described with reference to the system of the invention, configured to receive the measured level of sodium and optionally at least one medically relevant characteristic of the subject, calculate a subject-optimized dosage of a diuretic based the measured level of sodium and optionally the at least one medically relevant characteristic of the subject; and provide the calculated subject-optimized dosage of diuretic.

In some embodiments, the calculation of the subject-optimized dosage of the diuretic is based on the at least one medically relevant characteristic of the subject. In some embodiments, the calculation of the subject-optimized dosage of the diuretic is not based on the at least one medically relevant characteristic of the subject.

According to some embodiments, the device for collecting a urine sample is a urine-collection cup. According to some embodiments, the urine-collection cup is further configured to measure a urine output volume. In some embodiments, the urine-collection cup contains at least 200 ml. According to some embodiments, the device for collecting a urine sample is a condom-like catheter. According to some embodiments, the device is a diaper or a pad. According to some embodiments, the device for collecting a urine sample comprises electronic means for transmitting signal and/or data indicating the measured urine sodium level to a processing unit.

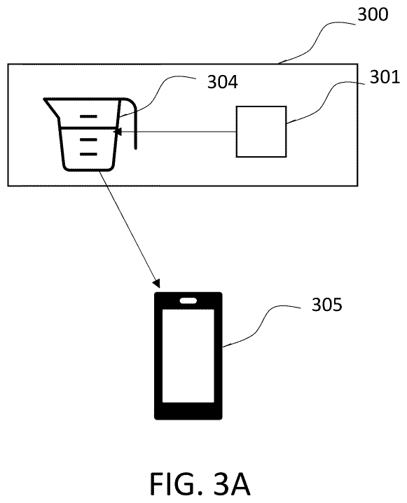

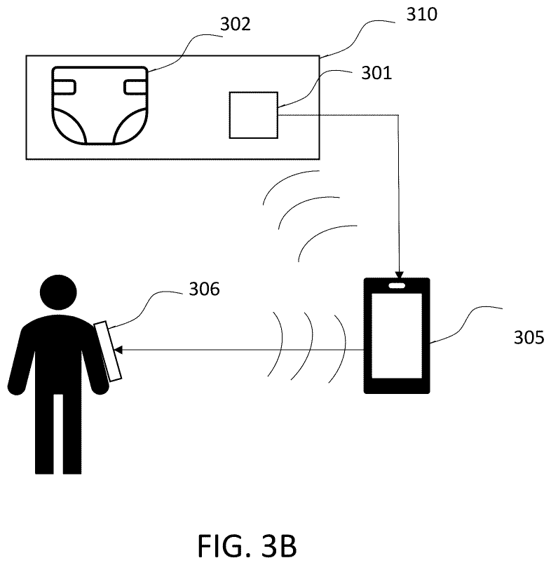

Reference is now made to FIGS. 3A-3B which schematically show examples of a kit for optimizing a dosage of a diuretic to a subject suffering from or at risk of heart failure, according to some embodiments. As shown in FIG. 3A, kit 300 comprises a sodium measurement sensor 301 that may be a chemo-electrical sensor, and a urine-collection cup 304. The urine-collection cup 304 collects a urine sample of the subject and the sodium measurement sensor may be manually inserted into urine-collection cup 304 with the urine sample of the subject, or be embedded in the urine-collection cup, in order to measure the level of sodium in the urine sample of the subject. In addition, urine-collection cup 304 may also serve for measuring the urine output volume. The sodium level measured in the urine sample with the urine-collection cup 304 may be manually inputted to the processing unit optionally together with at least one medically relevant characteristic using the user interface of smartphone 305, which represents a touch screen, or any other user interface provided with smartphone 305. Smartphone 305, which comprises the processing unit, configured to execute the calculation. The processing unit calculates the optimized dosage of diuretic recommended for the subject based on the measured level of sodium in the urine sample of the subject and optionally the at least one medically relevant characteristic, and the calculated dosage of diuretic recommended to the subject is presented on the screen of smartphone 305. Alternatively, the recommended dosage of diuretic may also be presented to the subject via a sound indicator indicating the subject the recommended dosage of diuretic. In addition, according to some embodiments, the urine-collection cup also serves for measuring the urine output volume of the urine sample which may be manually inputted to the processing unit by the subject using the user interface of smartphone 305.

In FIG. 3B, kit 310 comprises sodium measurement sensor 301 that may be a chemo-electrical sensor, and a diaper 302. Diaper 302 may be a smart diaper, which incorporates various sensors. The sodium measurement sensor 301 may be embedded in diaper 302. Alternatively, sodium measurement sensor 301 may be provided separately from diaper 302 and it may be manually mounted in diaper 302 by the subject. Once the urine sample of the subject is collected in diaper 302, sodium measurement sensor 301 measures the level of sodium in the urine sample and transmits, for example by electronic means, the measured level of sodium to the processing unit. At least one medically relevant characteristic may also be inputted by the subject to the processing unit, for example by the user interface of smartphone 305, and the processing unit executes a code for optimizing the dosage of diuretic recommended to the subject based on the measured level of sodium and optionally the at least one characteristic of the subject. The processing unit instructs a delivery device 306 which is associated with the processing unit and which is connected to the subject, to administer the calculated dosage of diuretic to the subject. Delivery device 306 administers the subject-optimized dosage of diuretic to the subject.

Another example, not demonstrated in the figures, is a device embedded in a toilet (a toilet-based system). The device includes the sodium measurement sensor of the invention for measuring sodium levels, and has also a sensor for monitoring the output volume of urine. The device reports the sodium and urine output volume measurements to a processor, which may be, for example, installed on a smartphone. The processor calculates and provides a subject-optimized dosage to an interface, such as the smartphone screen, and the subject may take a number of pills as recommended by the processor. Alternatively, the interface is a delivery device, such as a subcutaneous pump, which administers to the subject a diuretic, e.g. furosemide, according to the subject-optimized dosage provided.

An additional example for the “closed loop” system, according to some embodiments, is provided herein, based on the 2021 ESC Guidelines for the diagnosis and treatment of acute and chronic heart failure, European Heart Journal (2021) 42.

According to the example, a subject suffering from HF is on an oral loop diuretic (furosemide), and took 20 mg of furosemide. The subject is using the toilet-based system described above, including a sodium measurement sensor and a volume sensor, which are equipped with means for wireless transmission. In addition, the first dosage of furosemide has been manually entered into the system by using a smartphone carrying an application based on the above description of the processing unit. 2 hours after taking the furosemide, the subject uses the toilet, and the toilet-based device reports a measurement of 40 mEq/L urinary sodium and a urinary output of 70 mL/h. The measurement is transmitted by the device to the subject's smartphone, which calculates and displays a subject-optimized daily dosage of 40 mg furosemide. The subject takes the diuretic, and 2 hours later uses again the toilet-based device, with a measurement of 60 mEq/L urinary sodium and a urinary output of 120 mL/h. The device again transmits the data to the smartphone, which calculates and displays a subject-optimized daily dosage of 40 mg furosemide again. Further measurements of the urinary sodium and output volume remain the same, and the subject-optimized dosage remains 40 mg daily.

In some embodiments, there is provided a computerized system for determining a dosage of a diuretic required by a subject (subject-optimized dosage) suffering from or at risk of heart failure (HF), comprising one or more processors configured to:
receive a measured level of urinary sodium of the subject;
calculate a subject-optimized dosage of the diuretic based on the measured level of urinary sodium and optionally at least one medically relevant characteristic of the subject; and
provide an output comprising at least the calculated subject-optimized dosage of diuretic.

In some embodiments, the subject-optimized dosage of the diuretic is calculated based on the measured level of urinary sodium and the at least one medically relevant characteristic. In some embodiments, the subject-optimized dosage of the diuretic is not calculated based on a medically relevant characteristic.

The computerized system described herein relates to the processing unit described above, and therefore all embodiments relevant for the processing unit also apply to the computerized system.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims which follow.

1.-29. (canceled)

30. A system for determining a dosage of a diuretic required by a subject (subject-optimized dosage) suffering from or at risk of heart failure (HF), the system comprising a sodium measurement sensor and one or more processors, wherein:
the sodium measurement sensor is a chemo-electrical sensor configured to measure a sodium level in a urine sample of the subject and to relay the measurement of the urinary sodium level to the one or more processors; and
the one or more processors are configured to:
receive at least a first measurement of the urinary sodium level of the subject,
calculate at least a first subject-optimized dosage of the diuretic based on at least the first measurement of urinary sodium level and optionally at least one medically relevant characteristic of the subject, and
provide an output comprising at least the first calculated subject-optimized dosage of diuretic.

31. The system of claim 30, wherein the sodium measurement sensor is configured to measure only sodium levels.

32. The system of claim 30, wherein the sodium measurement sensor comprises electronic means for transmitting signal and/or data indicating the urine sodium level to the one or more processors.

33. The system of claim 30, wherein the sodium measurement sensor is embedded in a urine-collection cup, a condom-catheter, a diaper, or a toilet system.

34. The system of claim 30, further comprising a volume sensor configured to measure urine output volume of the subject, and wherein the one or more processors is further configured to receive at least a first urine output volume measurement, and to further calculate the subject-optimized dosage based on at least the first urine output volume measurement.

35. The system of claim 30, further comprising one or more sensors configured to measure one or more urinary parameter selected from the group consisting of urine output volume, potassium level, chloride level, creatinine level, and osmolality, in the urine sample of the subject, and wherein the one or more processors is further configured to receive at least a first measurement of the one or more urinary parameter, and to further calculate at least the first subject-optimized dosage based on at least the first measurement of the one or more urinary parameter.

36. The system of claim 30, wherein the one or more processors is further configured to receive the at least one medically relevant characteristic of the subject.

37. The system of claim 30, wherein the at least one medically relevant characteristic is selected from the group consisting of: age, gender, weight, body mass index (BMI), date of last acute exacerbation, number of previous acute exacerbations, previous dose of diuretic, type of diuretic, and any combination thereof.

38. The system of claim 30, wherein the one or more processors is further configured to calculate the subject-optimized dosage of the diuretic by using a machine learning algorithm.

39. The system of claim 38, wherein the machine learning algorithm is trained on a data set comprising dosages of diuretic administered to a plurality of subjects suffering from or at risk of heart failure, and a plurality of attributes associated with each of the plurality of dosages, the plurality of attributes comprising urinary sodium levels and optionally at least one medically relevant characteristic of the plurality of subjects.

40. The system of claim 30, further comprising a user interface associated with the one or more processors, the user interface being configured to display at least the subject-optimized dosage of diuretics provided by the one or more processors, and/or to allow entering information into the processing unit.

41. The system of claim 30, further comprising a delivery device functionally associated with the one or more processors, wherein the processing unit is further configured to instruct the delivery device to deliver the subject-optimized dosage of the diuretic to the subject.

42. The system of claim 41, wherein the delivery device is selected from the group consisting of a subcutaneous drug pump and a smart drug dispenser.

43. The system of claim 30, wherein the processing unit is further configured to calculate at least a second subject-optimized dosage of diuretics based on at least a second measurement of urinary sodium level and optionally one or more urinary parameter selected from the group consisting of urinary output volume, potassium level, chloride level, creatinine level, and osmolality, and on at least a first dosage of the diuretic administered to the subject after the first measurement of urinary sodium level and before the second measurement of urinary sodium level.

44. The system of claim 43, wherein the at least one medically relevant characteristic further comprises a change in the urinary sodium level and/or in the one or more urinary parameter between the first measurement and the second measurement.

45. The system of claim 44, further comprising a urine-collecting device.

46. The system of claim 30, located in at-home or outpatient setting.
